(19) United States
(12) Patent Application Publication
(10) Pub. No.: US 2012/0052063 A1
(43) Pub. Date: Mar. 1, 2012

(54) AUTOMATED DETECTION OF BREAST CANCER LESIONS IN TISSUE

(75) Inventors: Rohit Bhargava, Urbana, IL (US); F. Nell Pounder, Slidell, LA (US); Rohith K. Reddy, Champaign, IL (US)

(73) Assignee: The Board of Trustees of the University of Illinois

(21) Appl. No.: 13/222,623

(22) Filed: Aug. 31, 2011

Related U.S. Application Data
(60) Provisional application No. 61/378,763, filed on Aug. 31, 2010.

(51) Int. Cl. A61K 39/395; G06K 9/00; A61P 35/00

(52) U.S. Cl. 424/133.1; 382/128

(57) ABSTRACT

The present disclosure relates to methods of analyzing breast tumor samples, for example as a means to determine whether the tumor is cancerous or benign. For example, it is shown herein that analysis of a Fourier transform infrared (FT-IR) spectroscopic image allows for automated detection of breast cancer or benign breast tumors with high accuracy.

### CROSS-REFERENCE TO RELATED APPLICATION

This application claims priority to U.S. Provisional Application No. 61/378,763 filed Aug. 31, 2010, herein incorporated by reference.

### FIELD

This application relates to methods of evaluating breast tissue samples, for example, using infrared spectroscopic imaging.

### BACKGROUND

The paradigm for cancer detection and diagnosis is rather similar for most solid tumors. As an example, consider breast cancer. Screening for breast cancer is routine (Smith et al. CA Cancer J Clin 60: 99-119, 2010) as treatment is largely effective for early-stage disease (Horner et al. (2009) SEER Cancer Statistics Review, 1975-2006, NCI. Bethesda, Md., seer.cancer.gov/csr/1975_{—}2006/, based on November 2008 SEER data submission). If an abnormality is observed upon screening, a biopsy is conducted (Carter D (2004) in Interpretation of Breast Biopsies, ed 4 (Lippincott Williams & Wilkins, Philadelphia), pp 37-50). A manual examination of the structure and organization of cells (histology) within the biopsy is the gold standard for diagnoses. The seemingly simple task of recognizing cancer in a biopsy requires expert human input, leading to significant healthcare implications. First, large numbers of false positives (Elmore et al., N Engl J Med 338:1089-1096, 1998) are a natural consequence of sensitive screening. Consequently, more than a million people undergo breast biopsies in the United States annually (Thomson Reuters In-Patient and Out-Patient

Views Market-Scan Database (2008)) and about 80% are not actually diagnosed with cancer (Parker et al. (1994) Radiology 193: 359-362). Pathologists are forced to distribute attention over all patients rather than focusing on those cases that are truly positive. In the meantime, patients waiting for a diagnosis (Simunovic et al. (2001) Can Med Assoc J 165: 421-425) exhibit biochemical signals of elevated distress (Lang et al. (2009) Radiology 250: 631-637) and psychologic sequelae (Schwartz et al. (2004) JAMA 291: 71-78 and Gibson et al. (2009) J Public Health (Oxf). 31:554-60). The suboptimal diagnostic process is a key reason (Lerman et al. (1990) Rev Med 19: 279-290) for substantially reduced screening compliance (Andrykowski et al. (2001) Breast Cancer Res Treat 69: 165-178) for this very segment of population that is at high risk (Carter et al. (1988) Am J Epidemiol 128: 467-477). Of those diagnosed with disease, the delay and variability (Raab et al. (2005) Cancer 104: 2205-2213 and Bueno-de-Mesquita et al. (2010) Ann Oncol 21: 40-47) in diagnoses may degrade the quality of care. Hence, technologies that can aid in efficient histologic assessment will help accelerate accurate clinical decisions and the pace of research. Addressing this need to competently aid a human in histopathologic assessments remains a scientific and technological challenge.

Imaging technology to address this challenge is attractive, since visual evidence readily relates to clinical practice and provides information in a compact form. Simple structural imaging (e.g., optical microscopy of stained tissue) coupled with manual recognition is standard practice (Rosen, Rosen's Breast Pathology, Third Edition). Unfortunately, variability in staining and the limited information content of H&E stains has not allowed for robust automation (Schulte (1991) Histochemistry 95: 319-328 and

Jafari-Khouzani and Soltanian-Zadeh (2003) IEEE Trans Biomed Eng 50: 697-704). More recently, molecular imaging has provided for some understanding of specific epitopes' roles in cancer progression (Kumar and Richards-Kortum (2006) Nanomedicine 1: 23-30) and added to classical structure-based pathology (Mankoff (2008) Breast Cancer Res 10(Suppl. 1): S3). While several immunohistochemical markers can confirm specific transformations related to disease (Bast et al. (2001) J Clin Oncol 19: 1865-1878 and Slamon et al. (2001) N Engl J Med 344: 783-792), no single marker exists for universal identification of breast tumors (Nielsen et al. (2004) Clin Cancer Res 10: 5367-5374). Another alternative to add molecular information, chemical imaging, is emerging in which the contrast arises from endogenous chemical constitution of the tissue (Committee on Revealing Chemistry through Advanced Chemical Imaging & National Research Council of the National Academies Visualizing Chemistry: The Progress and Promise of Advanced Chemical Imaging. (National Academies Press: Washington, D.C., 2006)). Chemical imaging can be thought to be the imaging extension of label-free spectroscopy for every pixel in an image or the enhancement of structural images with molecular composition. Magnetic resonance spectroscopic imaging (MRSI), for example, is the chemical imaging analogue of MRI (Kwock et al. (2006) Lancet Oncol 7: 859-868) while mass spectroscopic imaging (King (2005) Am J Respir Crit Care Med 172: 268-279) is the imaging counterpart of mass spectroscopy (Stoecki et al. (2001) Nat Med 7: 493-496).

Fourier transform infrared (FT-IR) spectroscopic imaging, similarly, is the imaging analogue of molecular vibrational spectroscopy and provides an alternative microscopy platform for histopathology (Levin and Bhargava (2005) Ann Rev Phys Chem 56: 429-474). The absorption spectrum in the mid-IR region is a chemical fingerprint that can uniquely identify molecular species and their local environment (Ellis and Goodacre (2006) Analyst, 131: 875-885) and is potentially attractive for cancer pathology (Andrus (2006) Technol Cancer Res Treat. 5:157-167) due to its ability to detect biochemical transformations without dyes or stains. Several early studies applied non-imaging IR spectroscopy to discern pre-malignant tumor markers (Malins et al. (1995) Cancer 75: 503-517) and metastatic DNA features (Malins et al. (1996) Proc Natl Acad Sci USA 93: 2557-2563) in human breast tumor tissue samples, cell lines, and xenografted cells (Fabian et al. (1995) Biospectroscopy 1: 37-45 and Jackson et al. (1995) Biochim Biophys Acta 1270: 1-6). While these non-imaging works supported the concept of monitoring cancer-related biochemistry with IR spectroscopy, they did not provide a tool for clinical translation. Further, these studies typically measured only a few spectra from a small number of samples without regard for tissue, patient or clinical heterogeneity, likely resulting in significant chance and bias contributions—pitfalls that are well-known in biomarker research (Ransohoff (2005) Nat Rev Cancer 5:142-149). Recent technological advances (Lewis et al. (1995) Anal Chem 67: 3377-3381) have made imaging instrumentation that routinely and rapidly provides high-quality data (Bhargava and Levin. (2005) in Spectrochemical Analysis Using Infrared Multichannel Detectors, eds Bhargava R and Levin I W (Blackwell Publishing Ltd., Oxford)), commercially available and widely accessible.

Imaging is attractive as it provides both morphological and biochemical information and appeals directly to clinicians. In addition, there are scientific reasons to use imaging for breast pathology. The first step in cancer diagnosis is to separate histologic units of tissue and examine specific cell types individually for markers of malignancy (Fabian et al. (2003) J Mol Struct 661:411-417 and Anderson et al. (2006) Cell Cycle 5:1240-1244). Hence, the use of FT-IR microscopy (Fabian et al. (2002) Biopolymers 67: 354-357) and multivariate spectral analyses were proposed to provide clinically relevant information (Shaw et al. (2000) J Mol Struct-Theochem 500:129-138; Diem et al. (2004) Analyst 129:880-885; Petibois and Deleris (2006) TRENDS Biotechnol 24:455-462; Anastassopoulou et al. (2009) Vib Spectrosc 51: 270-275). One of the first efforts involved a small cohort of 77 samples to classify tumors by grade and steroid receptor status (Jackson et al. (1999) Cancer Detect Prey 23: 245-253). In another early study, several thousand spectra from 25 breast cancer patients with fibroadenoma, ductal carcinoma in situ (DCIS), or invasive ductal carcinoma were employed for classification using an artificial neural network (ANN) (Fabian et al. (2006) Biochim Biophys Acta 1758:874-882) and cluster analysis. Other notable approaches involved the novel use of slides and staining, as practiced in clinical settings, to assure compatibility with current practice (Dukor et al. (2000) Inst Phys Conf Ser 165: 79-80). Unfortunately, the low sample numbers, uncertain tissue heterogeneity and lack of demonstrated reproducibility have precluded a statistically significant validation of the approach.

### SUMMARY

The present application provides methods of analyzing a breast tissue sample, for example to determine if the sample containing a breast tumor is a breast cancer or benign tumor. In certain examples the method is a method of diagnosing breast cancer or benign breast tumors. For example, the method can include segmenting an infrared spectroscopic image of a breast tissue sample into epithelium and stroma, thereby classifying epithelium pixels and stroma pixels. The epithelium pixels are segmented into cancerous or benign, thereby analyzing the sample. This allows for the determination that the sample is cancerous or benign. In some examples the method also includes obtaining the infrared image of the breast tissue sample, such as obtaining a Fourier transform infrared (FT-IR) spectroscopic image. In some examples, segmenting the breast sample image into epithelium and stroma includes determining from the image one or more metrics selected from the group consisting of spectral peak heights, ratios of peaks, peak areas and centers of gravity. In some examples, segmenting epithelium pixels into cancerous or benign includes determining a spatial analysis of epithelium pixels, for example using a nearest neighbor approach.

In some examples the method can further include treating a subject identified as having breast cancer.

In some examples the method can further include selecting a subject suspected of having breast cancer and obtaining the breast tissue sample from the subject.

Also provided are computer-readable storage medium having instructions thereon for performing the disclosed methods, such as methods of analyzing a breast tumor sample and diagnosing breast cancer or a benign breast tumor.

The foregoing and other objects and features of the disclosure will become more apparent from the following detailed description, which proceeds with reference to the accompanying figures.

### BRIEF DESCRIPTION OF THE DRAWINGS

FIGS. 1A-E. FT-IR spectroscopic imaging provides biochemical and spectral information without dyes or contrast agents. (A) H&E-stained images. (B) An absorbance image at 1080 cm⁻¹ highlights vibrational modes generally associated with nucleic acids and glycoproteins that are prevalent in epithelium. (C) Absorbance image at 1236 cm⁻¹ corresponds to vibrational modes that highlight RNA, protein and collagen-rich breast stroma. (D) Pixels corresponding to stroma or epithelium are marked on an absorbance image after comparison with the H&E-stained image. (E) Average spectra from 50,182 epithelial and 140,100 stromal pixels demonstrate significant biochemical differences between the two sub-classes of tissue.

FIGS. 2A-F. Automated breast histopathology is performed by spectral and spatial analysis. Spectral classification is performed using supervised pattern recognition by (A) acquiring FT-IR spectroscopic imaging data from a large set of patients, which is reduced (B) to a smaller metric set. (C) Comparisons with corresponding H&E-stained images and clinical diagnostic data are used to develop (D) frequency distributions for sub-classes for each spectral feature. (E) A Bayesian classifier is used to categorize each pixel as stroma or epithelium. (F) Spatial information from resulting histology images is used for pathology classification. (F) Accuracy of each process is determined by ROC analysis on training and independent validation data.

FIGS. 3A-D. Robust and accurate automated epithelium identification is demonstrated on breast TMA images. (A) A color-coded classified breast TMA identifies epithelium as green and stroma as magenta. This TMA represents the first of the five independent validation arrays. (B) An adjacent H&E stained section for this TMA is included for reference. (C) An ROC curve displaying the sensitivity and specificity trade-off for epithelial and stromal classification. The mean AUC value is computed as 0.967. (D) Individual color-coded classified spectral images and corresponding H&E stained tissue demonstrate excellent pixel-level histology segmentation.

FIGS. 4A-D. Robust automated cancer segmentation is demonstrated by spatial polling of classified spectral images. (A) H&E staining forms the gold standard for cancer diagnosis. This TMA contains cancer and adjacent normal cores, as indicated by the column colors. (B) Spectral histology images for an adjacent TMA section segment epithelium from surrounding stromal tissue. (C) The color-coded histology image is used to compute spatial metrics and the classification process is repeated to segment cancer and normal epithelium pixels. (D) TMA calibration and validation (one shown) ROC analysis indicates a human-competitive accuracy in tumor identification. Dashed lines represent the boundaries for a 95% confidence region.

FIG. 5 is a composite validation curve for detecting cancer in breast tissue constructed from six independent patient sets, as detailed in Table 2.

FIGS. 6-9 are flow diagrams showing examples of the methods.

FIGS. 10A-F show consideration of box sizes and 10 epithelium thresholds, resulting in 120 different options for classification. To evaluate the relative potential of each of these classifiers, the frequency of box selection vs. the selected epithelium threshold is plotted for each box size. These plots for box sizes of 4×4 pixels (25 μm2), 8×8 pixels (50 μm2), and 12×12 pixels (75 μm2) are displayed in A-C. These plots indicate that each of these classifiers demonstrate potential for cancer diagnosis, as these is no overlap in the cancer and normal standard deviations (represented as error bars) for any combination of box size and epithelium threshold. This is confirmed by histograms of the frequency distributions at epithelium thresholds of 0.2, 0.5, and 0.8 each box size (D-F). FIGS. 11A-D are graphs showing automated histology and pathology with only spectral metrics. (A) Spectral metrics provide accurate histologic segmentation of stroma and epithelium with AUC values of ˜1 for each tissue class. (B) This classification is reproducible in validation on separate tissue samples. (C) Spectral metrics demonstrate reduced discrimination in separating cancer and normal epithelium pixels, with an AUC of only 0.80. (D) Spectral metrics do not provide reproducible pathology discrimination, as demonstrated by an AUC of 0.55 in validation samples.

FIGS. 12A-E are graphs showing a linear-fit model for core-level pathology classification at a specific box width. The slope and offset are computed for each core for the least-squares linear fit for the plot of box frequency vs. epithelium threshold. An offset cumulative distribution function for cancer and normal TMA cores at box sizes of (A) 5×5 pixels and (B) 9×9 pixels indicates that the optimal y-intercept for separation of cancer the normal cores shifts to a lower threshold with increasing box size. A plot of offset vs. slope for box sizes of (C) 5×5 pixels and (D) 9×9 pixels demonstrates the possibility for highly accurate separation of cancer and normal TMA cores. (E) A plot of the core-level AUC using offset, slope, and a scatter plot of offset and slope for cancer and normal tissue discrimination indicates optimal segmentation at larger box sizes.

FIGS. 13A-B are graphs showing non-linear fit for core-level pathology classification at a specific epithelium threshold. Parameters A, B, and C for a quadratic polynomial fit for plots of fraction of boxes above a selected epithelium threshold vs. box size are computed for each TMA core and cancer and normal classes are plotted at a threshold of (A) 30% epithelium and (B) 70% epithelium to demonstrate that no significant improvement in class separation is possible with a more complex 3 dimensional non-linear model.

FIGS. 14A-D are graphs showing pathology classification with a single box. (A) The false positive box fraction in normal TMA cores and (B) false negative box fraction in cancer TMA cores for each box width indicates that high epithelium thresholds and/or small box widths are not optimal for cancer classification from a single box. (C) A plot of box-level AUC vs. box width indicates that AUC increases with box width but approaches a limit of 0.82 above a box size of 7×7 pixels. (D) The ROC curve for a box size of 7×7 pixels has an optimal operating point at a threshold of 20% epithelium.

### DETAILED DESCRIPTION

Unless otherwise explained, all technical and scientific terms used herein have the same meaning as commonly understood by one of ordinary skill in the art to which a disclosed invention belongs. Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. “Comprising” means “including”; hence, “comprising A or B” means “including A” or “including B” or “including A and B.” All references cited herein are incorporated by reference.

Breast Tumor: A neoplastic condition of breast tissue that can be benign or malignant. The most common type of breast cancer is breast carcinoma, such as ductal carcinoma. Ductal carcinoma in situ is a non-invasive neoplastic condition of the ducts. Lobular carcinoma is not an invasive disease but is an indicator that a carcinoma may develop. Infiltrating (malignant) carcinoma of the breast can be divided into stages (I, IIA, IIB, IIIA, IIIB, and IV). See, for example, Bonadonna et al., (eds), Textbook of Breast Cancer: A clinical Guide the Therapy, 3rd; London, Tayloy & Francis, 2006.

Exemplary therapies for breast cancer include surgery (e.g., removal of some or all of the tumor), hormone blocking therapy (e.g., tamoxifen), radiation, cyclophosphamide plus doxorubicin (Adriamycin), taxane (e.g., docetaxel), and monoclonal antibodies such as trastuzumab (Herceptin) or pertuzumab, or combinations thereof.

Cancer: Malignant neoplasm, for example one that has undergone characteristic anaplasia with loss of differentiation, increased rate of growth, invasion of surrounding tissue, and is capable of metastasis.

Control: A sample or standard used for comparison with an experimental or test sample (such as a breast sample). In some embodiments, the control is a normal sample obtained from a healthy patient (or plurality of patients), such as a normal breast sample or plurality of samples. In some examples, the control is a non-tumor tissue sample obtained from a patient diagnosed with breast cancer, such as normal breast tissue. In some embodiments, the control is a known benign breast tumor sample (or plurality of samples). In some embodiments, the control is a known benign breast cancer sample (or plurality of samples).

In some embodiments, the control is a historical control or standard reference value or range of values (such as a previously tested control sample(s), such as a known breast cancer, normal breast sample, benign breast sample, epithelium, or stroma). In some embodiments the control is a standard value representing the average value (or average range of values) obtained from a plurality of patient samples, such as known normal breast samples or known breast cancer samples. For example control samples can be used to determine a probability of distribution function (pdf) for a particular characteristic, such as a pdf for a known epithelium pixel or known stroma pixel (such as pdfs for particular matrices such as peak ratio), known breast cancer spatial pattern, or known benign breast tumor pattern.

Diagnose: The process of identifying a medical condition or disease, for example from the results of one or more diagnostic procedures. In particular examples, diagnosis includes determining whether a breast sample obtained from a subject is a breast cancer, or a benign breast tumor.

Normal cells or tissue: Non-tumor, non-malignant cells and tissue.

Sample: A sample, such as a biological sample, is a sample obtained from a subject. As used herein, biological samples include all clinical samples useful for detection of breast tumors, such as breast cancer or a benign breast tumor, in subjects. Samples include but are not limited to, cells, tissues, and bodily fluids, obtained from the breast such as: biopsied or surgically removed tissue, including tissues that are, for example, unfixed, frozen, fixed in formalin and/or embedded in paraffin, as well as milk. In a particular example, a sample includes breast tissue obtained from a human subject, such a biopsy sample, for example a fine needle aspirate, a core biopsy sample, or an excisional biopsy sample. In some examples, a breast tissue sample is a fresh sample, frozen sample, or fixed sample (e.g., embedded in paraffin).

Subject: Includes any multi-cellular vertebrate organism, such as human and non-human mammals (e.g., veterinary subjects). In some examples, a subject is one who has cancer, or is suspected of having cancer, such as breast or mammary cancer.

Suitable methods and materials for the practice and/or testing of embodiments of the disclosure are described below. Such methods and materials are illustrative only and are not intended to be limiting. Other methods and materials similar or equivalent to those described herein also can be used. For example, conventional methods well known in the art to which a disclosed invention pertains are described in various general and more specific references.

### Overview of the Technology

Histopathologic assessment of stained tissue is a cornerstone of contemporary clinical diagnoses and research in cancer. Manual assessments, however, can lead to increased cost, errors and diagnostic inconsistency; hence, analytical technologies that can compete with humans in histologic recognition are highly desirable. Provided herein is a human-competitive histopathologic recognition of breast cancer method that uses chemical imaging technology. Briefly, the method includes Fourier transform infrared (FT-IR) spectroscopic imaging to image biopsy sections without the use of dyes or stains. Subsequently, objective numerical algorithms are developed for accurate histologic and pathologic classification, without manual input. Since pathology is largely concerned with epithelial tumors, it was confirmed using coupled spectral-spatial statistical pattern recognition that the method could accurately segment tissue into epithelium and stroma, followed by segmentation of epithelial cells into cancer and normal classes. Rigorous statistical validation using receiver operating characteristic (ROC) analyses for over 800 samples drawn from different patient cohorts demonstrated that the burden of false positives may be reduced for 80% of patients with minimal error. Pre-pathologist triaging of samples can also be efficiently accomplished to aid in accurate and rapid decision-making. Clinical translation of this technology can substantially reduce the burden of mammographic screening on patients and on the healthcare system.

Providing rapid, accurate and reproducible histologic diagnoses that lead to effective healthcare at affordable cost are of contemporary interest to clinicians, pathologists, insurance sector, and in public health. While prior studies have indicated that FT-IR imaging has the potential to address this clinical need, it has not been adopted for automated histopathology clinically due to a variety of factors. A primary reason is the lack of robustly validated protocols that address a key clinical question. Provided herein are methods that use IR images to diagnose breast cancer.

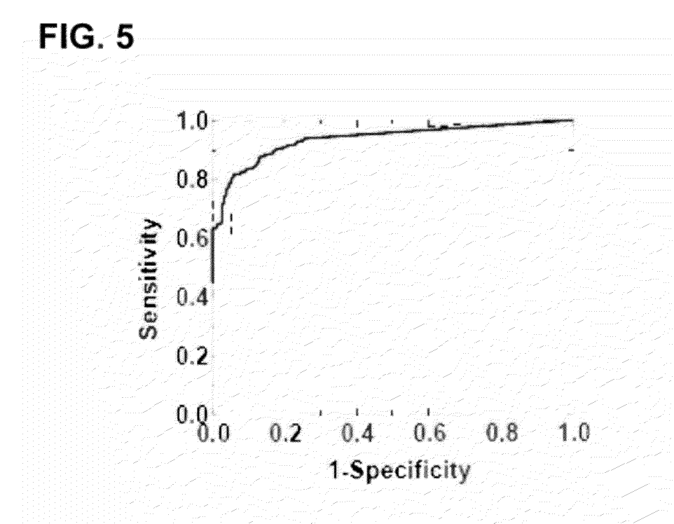

The disclosed methods can be used in combination with the human element of diagnosis (e.g., a pathologist) to produce accurate results in an efficient manner that benefits both the healthcare enterprise and the patient. For example, from a typical university hospital practice, breast cancer diagnosis was found to have a sensitivity of ˜97% (37 missed tumors in 1102 samples) (Wiley and Keh (1999) Am J Surg Pathol 23: 876-884). Hence, it was sought to assure that the results of the disclosed methods are capable of achieving similar performance. The validation statistics were employed to determine, first, an operating point (sensitivity, specificity) from the composite validation ROC curve (FIG. 5). An operating point of 95% sensitivity was selected, for which the specificity is 82.5%. As 80% of breast biopsies are benign (Parker et al. (1994) Radiology 193: 359-362), the disclosed methods permit a rapid intimation for ˜80% of benign (˜650,000 women per year in the US alone) without significant additional errors.

Using the disclosed methods in conjunction with human decision-making, a second measure of benefit is how the method improves sample triage for pathologists, which can be quantified by the likelihood ratio (LR). LRs measure the power of a test to change the pre-test into the post-test probability of a disease being present (Fagan (1975) N Engl J Med 293257; Jaeschke (1994) JAMA 271:703-707). For the selected sensitivity and specificity and for the prevalence of disease in biopsy samples being 0.2, the LR of a positive test (LR+) is ˜5.4 (4.65-6.33, 95% confidence interval (CI)), indicating that the pool of samples at risk of disease can be enriched from 20% with cancer to 58% (54%-61%, 95% CI) if the method is used between screening and pathologist examination. The LR of the negative test (LR-; i.e., to rule out disease), is ˜0.06 (0.03-0.11, 95% CI), indicating that the presence of disease in the samples labeled benign by the disclosed methods is reduced to less than 1% (0.8-3%, 95% CI). Hence, the application of spectroscopic triaging using the disclosed methods can improve the accuracy and efficiency of pathology practice.

Statistical validation was used to demonstrate the benefits of the disclosed methods. The disclosed methods provide 95% confidence intervals for the employed sensitivity and specificity as 95.0±1.8% and 82.5±5.7% respectively. The benefits of the large sample size of this study become apparent in examining the CI. While greatly diminishing returns are seen for larger sample sizes, e.g. ±3% CI for 600 control samples for specificity, catastrophic effects are seen for smaller samples, e.g. ±15% for 25 samples. Similarly, the sensitivity CIs are only reduced to ±1.3% in increasing cancer samples to 1000 but the CIs for tens of samples increase substantially (e.g. ±8.5% for 25 samples). One aspect of validation is the number of samples used to establish this sensitivity. To claim that the accuracy of 95% lies at the lower limits of the 95% confidence interval of a test with accuracy of 98% with a probability of at least 0.95, over 500 cases are needed (Flahault et al. (2005) J Clin Epidemiol 58:859-862) for the power of the study to be at least 0.9. For 50 samples at the same power, for example, the lower CI would be 85%, which is considerably lower than human accuracy. Hence, the larger sample size here demonstrates the human-competitive results and confidence in those results at the common values of Type I and II errors used to evaluate diagnostic tests.

Thus provided herein are automated methods for determining whether a breast sample is cancerous or not. The results indicate that translation to clinical practice can be undertaken and there will be tangible benefits for both clinicians and patients in addressing the largest cancer in women. While this rapid preliminary diagnosis after a biopsy is important for screening follow-up, eliminating the need for human supervision and staining of samples is a novel avenue to evaluate surgical resections intra-operatively. The results herein demonstrate the potential of IR imaging which is relevant to public health and is a major step in the continuing progress of spectroscopic imaging towards clinical translation.

### Methods of Screening Breast Samples

Histopathologic assessment of stained tissue is a cornerstone of contemporary clinical diagnoses and research in cancer. Manual assessments, however, can also lead to increased cost, errors and diagnostic inconsistency; hence, analytical technologies that can compete with humans in histologic recognition are highly desirable. Provided herein is a human-competitive histopathologic recognition of breast cancer using emerging chemical imaging technology. In some examples, Fourier transform infrared (FT-IR) spectroscopic imaging is used to image biopsy sections (for example without the use of dyes or stains, such as H&E). Subsequently, objective numerical algorithms are used for accurate histologic and pathologic classification, without manual input. Such methods are suited to addressing a cause of the high emotional and economic burden of breast cancer screening.

The concept of using chemical imaging to examine large numbers of biopsies upon population screening is validated herein. Since pathology is largely concerned with epithelial tumors, it is shown herein that the method can demonstrate highly accurate segmentation of tissue into epithelium and stroma, followed by segmentation of epithelial cells into cancer and normal classes using coupled spectral-spatial statistical pattern recognition. Rigorous statistical validation using receiver operating characteristic (ROC) analyses for over 800 samples drawn from different patient cohorts demonstrates that the burden of false positives may be reduced for 80% of patients with minimal error. Pre-pathologist triaging of samples can also be efficiently accomplished to aid in accurate and rapid decision-making. Clinical translation of this technology can substantially reduce the burden of mammographic screening on patients and on the healthcare system.

The present application provides methods for analyzing a breast tumor sample, for example from a subject suspected of having breast cancer or a benign breast tumor. Thus, in some examples the methods can be used to distinguish breast cancer from a benign breast tumor, thereby permitting diagnosis of breast cancer or a benign breast tumor. In some examples, subjects suspected of having breast tumors (such as a breast cancer or benign tumor) are selected, and a breast tissue sample obtained (such as a biopsy sample). In some examples, if the sample is determined to be positive for breast cancer, the sample is selected for further analysis, for example additional analysis by a pathologist, or additional diagnostic procedures can be applied (such as additional histopathologic testing). In some examples, if the sample is determined to be positive for breast cancer, the subject is selected for treatment of the breast cancer, such as surgical resection of the cancer or breast; radiation therapy, or chemotherapy, or combinations thereof. Such treatments are known in the art. The disclosed methods are suitable for both human and mammalian veterinary subjects that may have a breast or mammary tumor.

In particular examples the method includes segmenting an infrared image of a breast tissue sample into epithelium and stroma, thereby classifying epithelium pixels and stroma pixels. The resulting epithelium pixels are segmented into cancerous or benign, thereby analyzing the sample. The method can also include obtaining the infrared image of the breast tissue sample, such as a Fourier transform infrared (FT-IR) spectroscopic image of the breast sample.

The method can also include preparing the breast tissue samples for such imaging using routine methods. Methods of processing a breast tissue sample for IR spectroscopic analysis are routine in the art. For example, the tissue can be fixed, fresh, frozen, paraffin embedded, or combinations thereof. In some examples, the image is obtained from an unstained sample, or from a sample stained with H&E.

In particular examples, segmenting the breast sample image into epithelium and stroma includes determining from the image one or more metrics. For example, one or more of spectral peak heights, ratios of peaks, peak areas and centers of gravity can be determined for a plurality of pixels of the IR image. Particular examples are shown in Table 1. For example, for each pixel a peak ratio of positions 1080:1456 cm⁻¹, 1556:1652 cm⁻¹, 1080:1238 cm⁻¹, and 1338:1080 cm⁻¹, a center of gravity of position 1216-1274 cm⁻¹, and a peak area of position 1426-1482 cm⁻¹ can be determined. Thus, in this example, for each pixel, six metrics are identified and assigned a value.

To determine whether the pixel is to be assigned stroma or epithelium, the value for each metric can be compared to a probability distribution function (pdf) for reference epithelium and stroma. Reference pdfs for epithelium and stroma can be determined using control samples, such as known breast cancer or benign breast tumor samples (see for example F. N. Keith, “Automated breast histopathology using MID-FTIR imaging”, Thesis (M.S.), University of Illinois at Urbana-Champaign, 2007). For example, reference pdf values for epithelium and stroma can be determined using pixels known to contain stroma or epithelium using H&E staining. Large numbers of control pixels can be assigned and used to obtain reference values or ranges of values for each metric. Such reference values can be compared to experimental values obtained for each metric. In some examples, there is overlap between the reference pdf values for stroma or epithelium for one or more metrics. In this case, a probability of stroma or epithelium is assigned, and a determination is made upon comparing all of the metrics. Thus by extracting from the image one or more metrics, this permits pixels of the breast sample image to be assigned as epithelium or stroma.

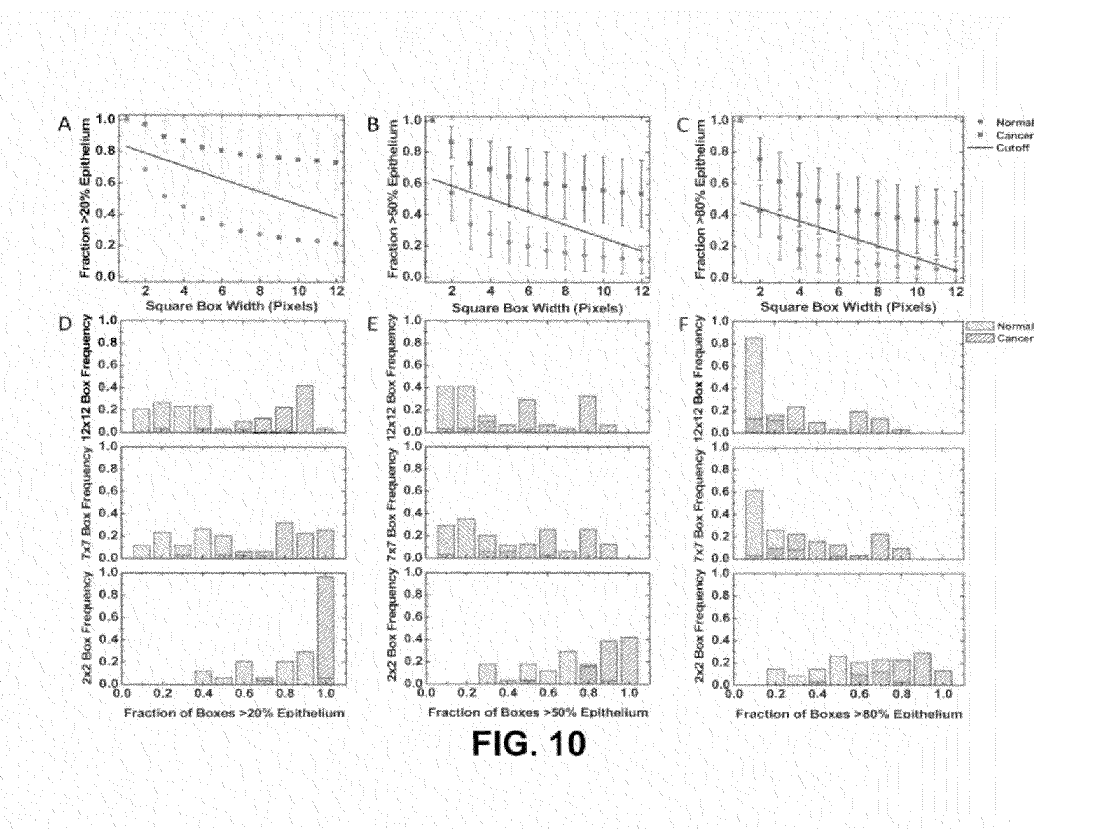

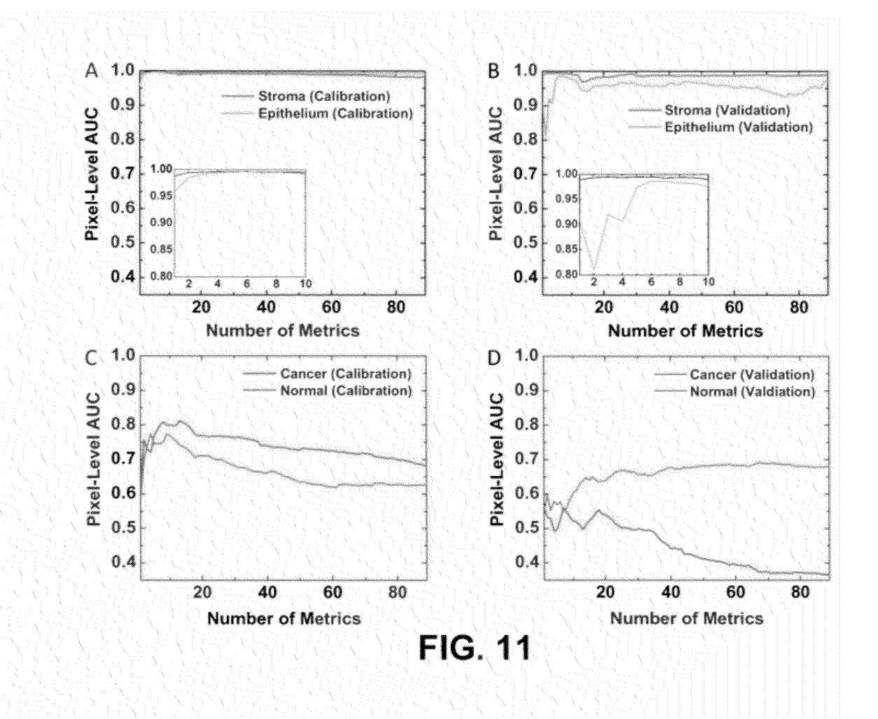

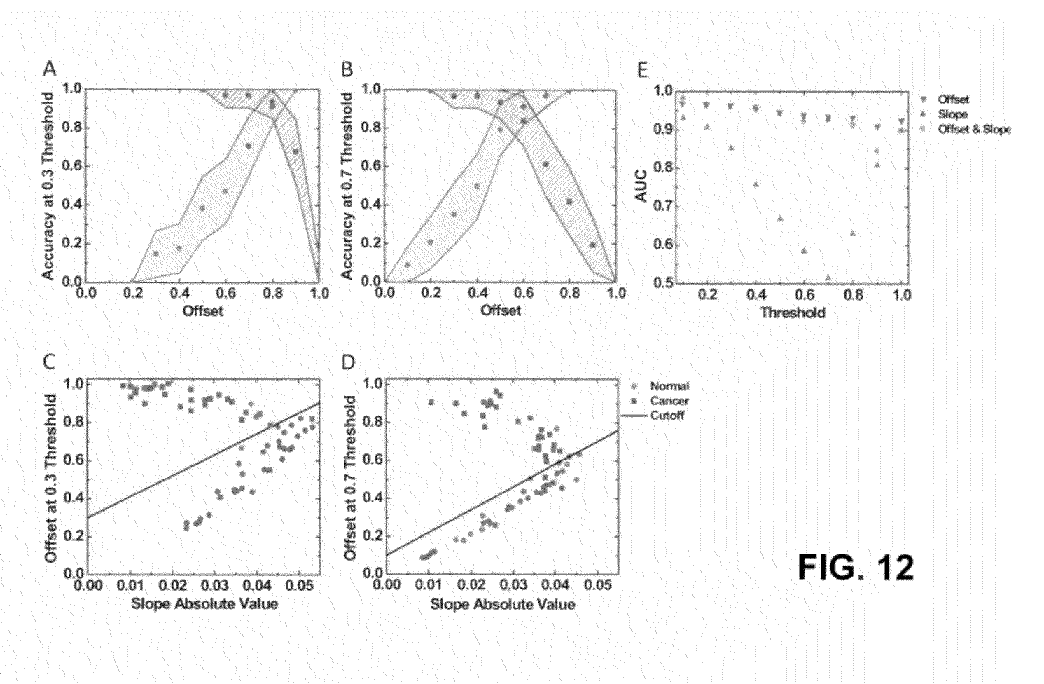

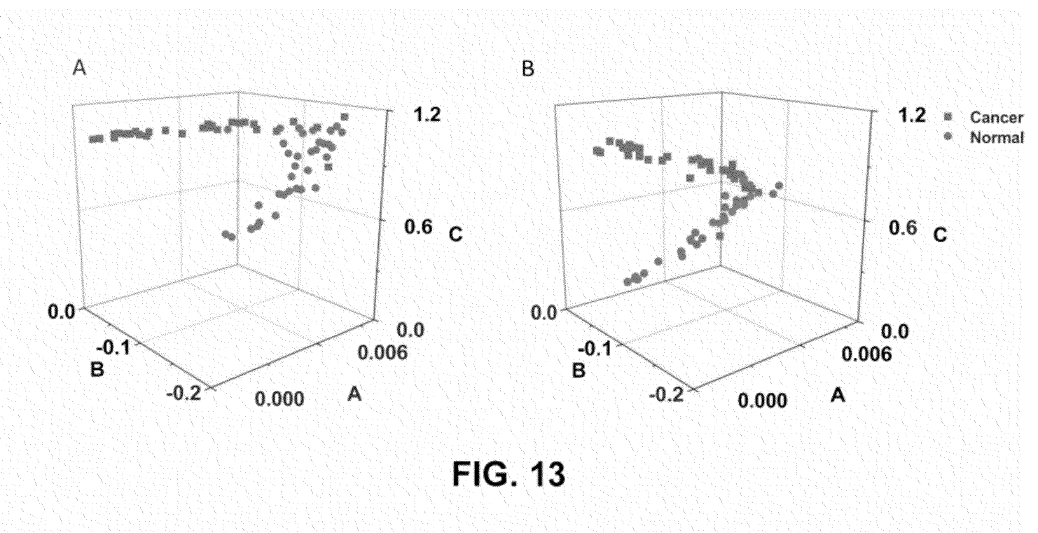

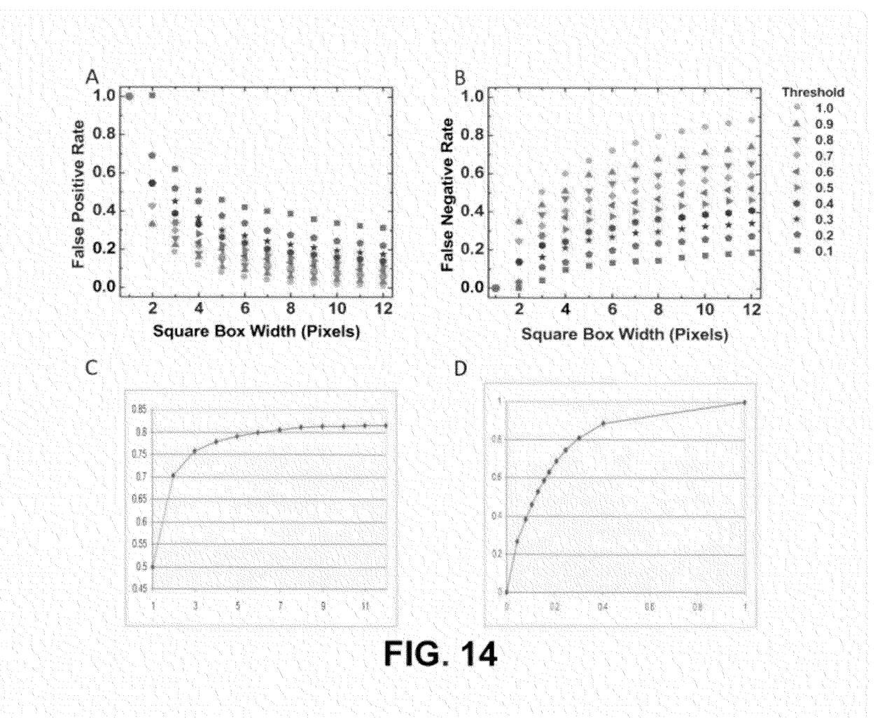

In particular examples, segmenting the epithelium pixels into cancerous or benign includes determining a spatial analysis of epithelium pixels. For example, the pixels assigned as epithelium can be further analyzed to determine if the epithelial spatial pattern in the image is cancerous or benign. Exemplary spatial analysis of epithelium can include examining the epithelium pixel density and neighborhood patterns. For example, the spatial neighborhood of a single epithelium pixel can examined progressively by increasing distance for prevalence and spatial distribution of epithelial and stromal cells as well as empty space. To determine whether the sample is to be assigned cancer or benign, the spatial pattern determined for the experimental sample can be compared to a probability distribution function (pdf) for reference cancer and benign epithelium special patterns. Reference pdfs for cancer and benign tumor can be determined using control samples, such as known breast cancer or benign breast tumor samples (for example, see FIGS. 10-14). For example, reference pdf values for spatial epithelium patterns can be determined using IR images from known breast cancer and benign tumor samples. Large numbers samples can be assigned and used to obtain reference pdf values or ranges of values. Such reference pdf values can be compared to experimental values. Thus by determining the spatial pattern of epithelium from the image and comparing to reference pdf values, this permits the breast sample image to be assigned as cancerous or benign.

In some embodiments, once a sample is analyzed, an indication of that analysis can be displayed and/or conveyed to a clinician or other caregiver. For example, the results of the test can be provided to a user (such as a clinician or other health care worker, laboratory personnel, or patient) in a perceivable output that provides information about the results of the test. In some embodiments, the output is a paper output (for example, a written or printed output), a display on a screen, a graphical output (for example, a graph, chart, voltammetric trace, or other diagram), or an audible output.

In other embodiments, the output is a diagnosis, such as whether the test breast sample analyzed is cancerous or benign. In additional embodiments, the output is a graphical representation, for example, a graph that indicates the value (such as amount or relative amount) of the likelihood that the sample is cancerous or benign. In some examples, the output is a number on a screen/digital display indicating the probability of the sample being cancer. In some examples, the output is text, indicating the likelihood that the sample is cancerous or benign along with the corresponding implications to the patient. Sensitivity, specificity, and confidence intervals may also be a part of the output. These outputs can be in the form of graphs or tabulated numbers. The output can be a color-coded image (e.g., of tissue cores) with different colors indicating different probabilities of being cancer or normal. In some embodiments, the output is communicated to the user, for example by providing an output via physical, audible, or electronic means (for example by mail, telephone, facsimile transmission, email, or communication to an electronic medical record).

In some embodiments, the output is accompanied by guidelines for interpreting the data, for example, numerical or other limits that indicate whether the test sample is cancerous or benign. The guidelines need not specify whether the test sample is cancerous or benign, although it may include such a diagnosis. The indicia in the output can, for example, include normal or abnormal ranges or a cutoff, which the recipient of the output may then use to interpret the results, for example, to arrive at a diagnosis, prognosis, or treatment plan. In other embodiments, the output can provide a recommended therapeutic regimen. In some embodiments, the test may include determination of other clinical information (such as determining the amount of one or more additional biomarkers in the biological sample).

In particular examples, the methods provided herein have a sensitivity of at least 90%, at least 95%, at least 98%, at least 97%, or at least 99% sensitivity, wherein sensitivity is the probability that a statistical test will be positive for a true statistic. In particular examples, the methods provided herein have a specificity of at least 70%, at least 75%, at least 80%, at least 82%, at least 85% or at least 90% specificity, wherein specificity is the probability that a statistical test will be negative for a negative statistic.

Also provided herein are computer-readable storage medium having instructions thereon for performing a method of analyzing a breast tumor sample, for example to diagnose the sample as breast cancer or a benign breast tumor. Thus, computer-readable storage medium having instructions thereon for performing the methods described herein are disclosed.

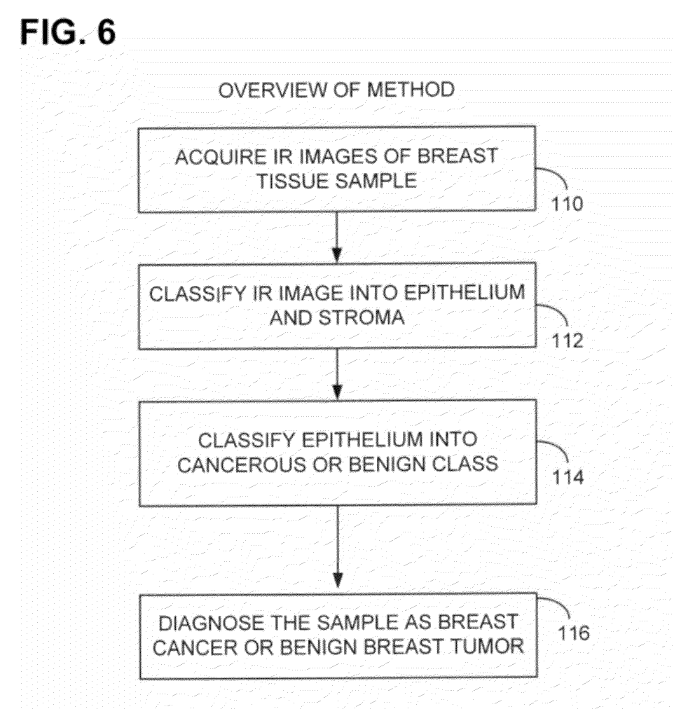

FIGS. 6-9 illustrate a method for analyzing a breast tumor sample, for example as a means to diagnose breast cancer or a benign breast tumor. Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

Any of the disclosed methods can be implemented as computer-executable instructions stored on one or more computer-readable media (e.g., non-transitory computer-readable media, such as one or more optical media discs, volatile memory components (such as DRAM or SRAM), or nonvolatile memory components (such as hard drives)) and executed on a computer (e.g., any commercially available computer, including smart phones or other mobile devices that include computing hardware). Any of the computer-executable instructions for implementing the disclosed techniques as well as any data created and used during implementation of the disclosed embodiments can be stored on one or more computer-readable media (e.g., non-transitory computer-readable media). The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, JavaScript, IDL, Matlab, Adobe Flash, or any other suitable programming language Likewise, the disclosed technology is not limited to any particular computer or type of hardware. Certain details of suitable computers and hardware are well known and need not be set forth in detail in this disclosure.

The disclosed methods, apparatus, and systems should not be construed as limiting in any way. Instead, the present disclosure is directed toward all novel and nonobvious features and aspects of the various disclosed embodiments, alone and in various combinations and subcombinations with one another. The disclosed methods, apparatus, and systems are not limited to any specific aspect or feature or combination thereof, nor do the disclosed embodiments require that any one or more specific advantages be present or problems be solved.

Turning to FIG. 6, in process block 110, IR images of a breast tissue sample (such as one containing a tumor or portion thereof) are acquired. For example, FT-IR images of a breast tissue sample can be taken directly, or obtained from another source. In process block 112, the IR images re classified. The classification process is used to classify the data into epithelium and stroma. For example, the classification can be used to segment stroma from epithelium, such as designating particular pixels of the IR image as stroma and others as epithelium. In process block 114, the pixels designated as epithelium in process block 112 are further classified as cancerous or benign. In process block 116, the breast tissue sample designated as being cancerous or benign. For example, the epithelium pixels can be analyzed for their epithelium content and/or spatial organization.

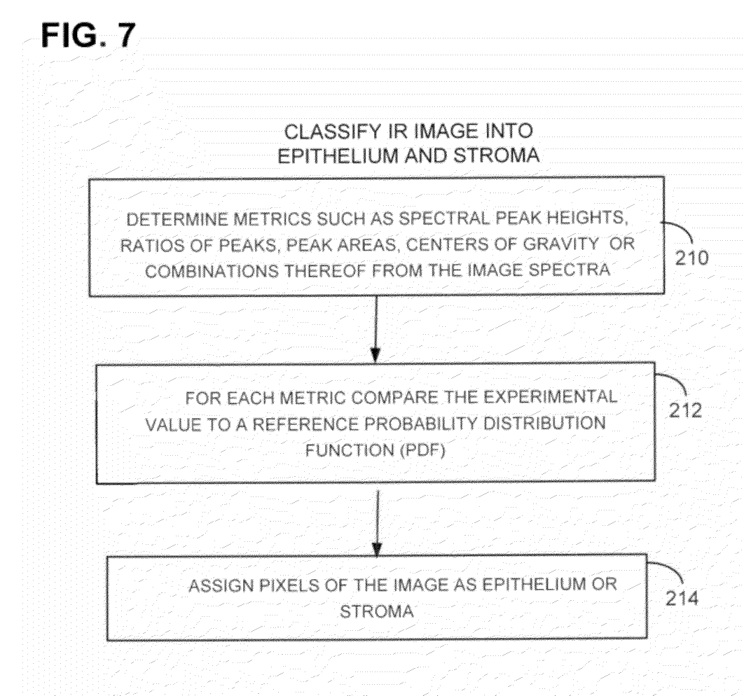

FIG. 7 is a flowchart of a method showing an example of how stroma can be segmented from epithelium in process block 112. In process block 210, the spectra from the images are analyzed for classification, such as by determining a plurality of metrics, for example spectral peak heights, ratios of peaks, peak areas and centers of gravity, or combinations thereof, for each pixel. In process block 212, the experimental value determined for each metric in process block 210 is compared to a reference probability distribution function (pdf). In process block 214, pixels of the image are assigned as either stroma or epithelium.

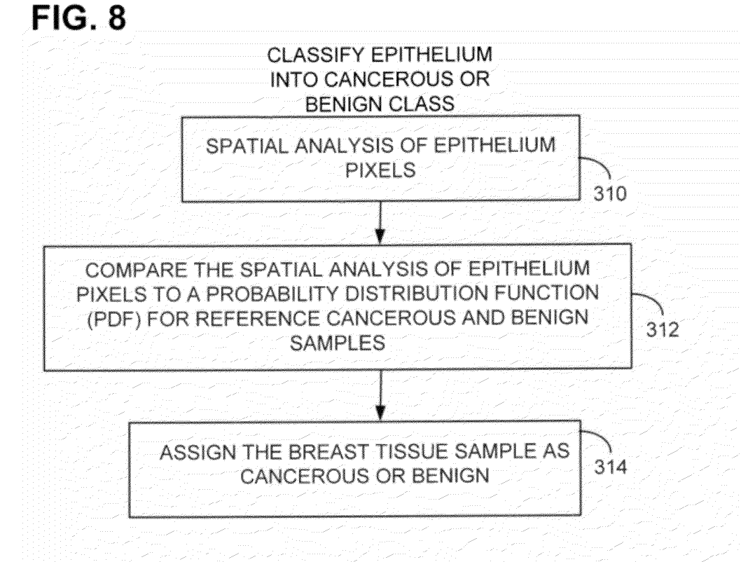

FIG. 8 is a flowchart of a method showing an example of classifying the epithelium as cancerous or benign in process block 114. In process block 310, a spatial analysis of the epithelium pixels and its neighborhood (i.e., other cell types, empty space) is performed. In process block 312, the experimental spatial analysis determined in process block 310 is compared to a reference probability distribution function (pdf) for reference cancerous and benign samples. In process block 314, the sample is assigned as cancerous or benign tumor.

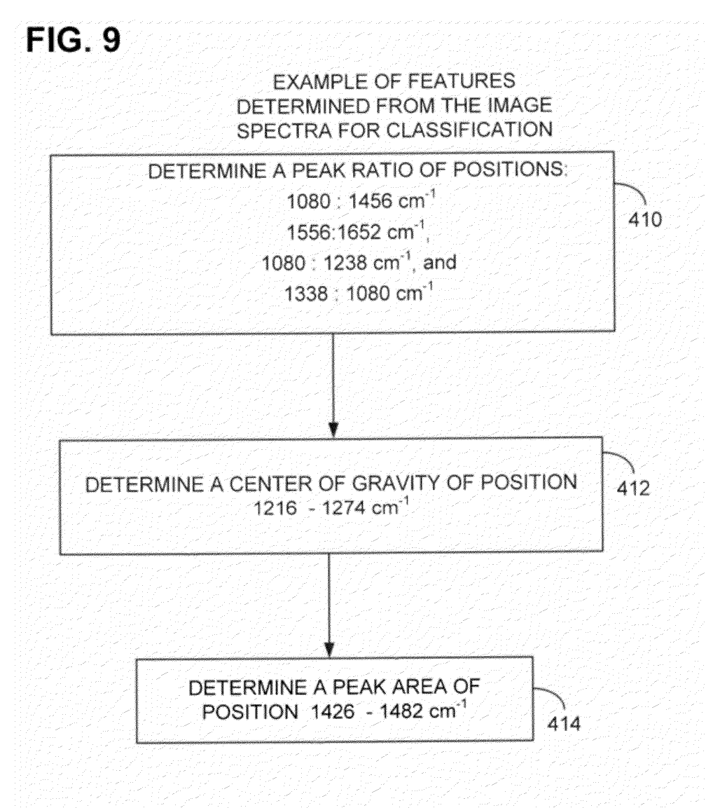

FIG. 9 is a flowchart of a method showing an example of determining particular features (metrics) for classification from the IR image (process blocks 114 and 210). There are a variety of features that can be determined. FIG. 9 shows some examples of features that can be extracted from the IR image, and used to classify a pixel as epithelium or stroma. In process block 410, the peak ratio of positions 1080:1456 cm¹, 1556:1652 cm⁻¹, 1080: 1238 cm⁻¹, and 1338: 1080 cm⁻¹ can be determined. In process block 412, a center of gravity of position 1216-1274 cm⁻¹ can be determined. In process block 414, the peak area of position 1426-1482 cm⁻¹ can be determined.

### Biological Samples

Disclosed methods can be performed using biological samples obtained from breast tissue, for example from any subject suspected of having breast cancer, a benign breast tumor, or a mammary tumor. A typical subject is a human female; however, any mammal that has a mammary tissue that may develop cancer can serve as a source of a biological sample useful in the disclosed methods. Exemplary biological samples useful in a disclosed method include tissue samples (such as breast tissue biopsies containing a tumor), such as can be collected by fine needle aspirates or core biopsies.

Samples may be fresh or processed post-collection (e.g., for archiving purposes). In some examples, processed samples may be fixed (e.g., formalin-fixed) and/or wax- (e.g., paraffin-) embedded. Fixatives for mounted cell and tissue preparations are well known in the art and include, without limitation, 95% alcoholic Bouin's fixative; 95% alcohol fixative; B5 fixative, Bouin's fixative, formalin fixative, Karnovsky's fixative (glutaraldehyde), Hartman's fixative, Hollande's fixative, Orth's solution (dichromate fixative), and Zenker's fixative (see, e.g., Carson, Histotechology: A Self-Instructional Text, Chicago:ASCP Press, 1997).

In some examples, the breast tissue sample (or a fraction thereof) is present on a solid support. Solid supports useful in disclosed methods need only bear the biological sample and, optionally, but advantageously, permit the convenient detection of components (e.g., stroma, epithelial cells) in the sample. Exemplary supports include microscope slides (e.g., glass microscope slides or plastic microscope slides), specialized IR reflecting or transmitting materials (e.g., BaF₂ slides or reflective slides), coverslips (e.g., glass coverslips or plastic coverslips), tissue culture dishes, multi-well plates, membranes (e.g., nitrocellulose or polyvinylidene fluoride (PVDF)) or BIACORE™ chips.

### Control Samples

In some methods, the experimental values determined from the breast tissue sample are compared to a standard value or a control sample, such as a probability distribution function (pdf) value (or range of values) for reference or control samples. A standard value or range of can include, without limitation, the pdf value or range of values for metrics (such as spectral peak heights, ratios of peaks, peak areas and centers of gravity of the IR image, for example, a peak ratio of positions 1080:1456 cm⁻¹, 1556:1652 cm⁻¹, 1080:1238 cm⁻¹, and 1338:1080 cm⁻¹, a center of gravity of position 1216-1274 cm⁻¹, and a peak area of position 1426-1482 cm⁻¹) for stroma and for epithelium. A standard value or range of can include, without limitation, the pdf value or range of values for the spatial pattern of epithelium pixels for breast cancer and for benign breast tumor. Such values can be obtained from a patient or patient population in which it is known that breast cancer or a benign breast tumor was present. A control sample can include, for example, normal breast tissue or cells, breast tissue or cells collected from a patient or patient population in which it is known that a benign breast tumor was present, or breast tissue or cells collected from a patient or patient population in which it is known that breast cancer was present.

### Example 1

### Materials and Methods

This example describes the materials and methods used in Examples 2-5.

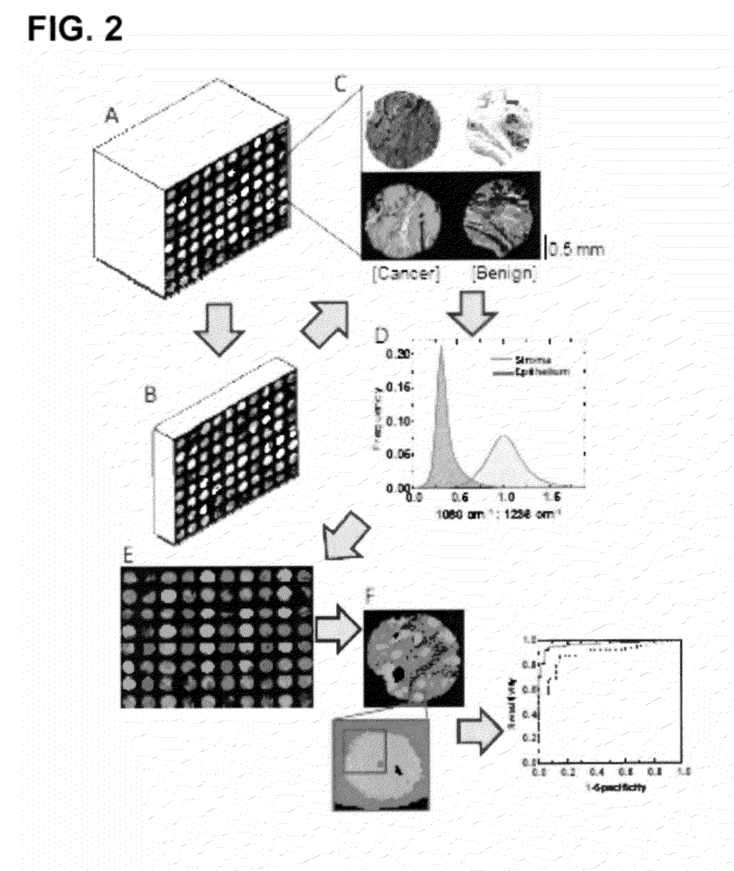

Materials: Seven TMAs, consisting of over 800 tissue samples from over 700 patients were analyzed (US Biomax Inc.). The TMAs consist of formalin-fixed, paraffin-embedded tissue cores that are sectioned onto barium fluoride (BaF₂) substrates to permit data collection over the entire mid-IR spectral region of interest (4000-720 cm⁻¹). The first sample set contains carcinoma and adjacent normal tissue from 37 patients in the form of 1.5 mm diameter cores on a single TMA. After pathologist evaluation, cores for 3 patients were eliminated due to inconclusive diagnosis. The cores from the remaining 34 patients (1 invasive lobular carcinoma, 33 invasive ductal carcinomas) were used as a calibration data set to develop algorithms to segment breast histology and pathology as outlined in FIGS. 2A-F.

These algorithms are then validated on a second copy of the TMA containing different tissue sections from the same patients and subsequently validated on five independent TMAs with 1 mm diameter cores from separate sets of patients. These TMAs contained 199 cores (120 invasive ductal carcinoma, 19 invasive lobular carcinoma, 20 normal, 14 adjacent normal, and 26 inconclusive diagnoses due to insufficient epithelium), 182 cores (77 invasive ductal carcinoma, 78 invasive lobular carcinoma, 1 mixed ductal/lobular carcinoma, 5 normal, 19 adjacent normal, and 2 inconclusive due TMA core damage), 82 cores (50 invasive ductal carcinoma, 2 invasive lobular carcinoma, 6 medullary carcinoma, 4 tubular carcinoma, 2 mucinous carcinoma, 10 hyperplasia, and 8 normal), 91 cores (36 invasive ductal carcinoma, 9 lymph node metastases, 2 hyperplasia, 34 adjacent normal breast, and 10 normal), and 146 cores (126 invasive ductal carcinoma, 8 invasive lobular carcinoma, 4 ductal carcinoma in situ, 2 Paget's disease, and 6 normal/hyperplasia).

Prior to infrared imaging, paraffin is removed from each TMA by immersion in hexane with stifling for 48-72 hours at 40° C. To ensure continued paraffin removal, fresh hexane is added every 3-4 hours. Paraffin elimination is checked every 24 hours on several tissue cores to monitor the disappearance of the 1462 cm⁻¹ peak.

Data Acquisition: A Perkin-Elmer Spotlight 400 FT-IR imaging spectrometer is used for data collection at a 6.25 μm pixel size and a 4 cm⁻¹ spectral resolution with 2 scans per pixel. A coarser spectral resolution of 16 cm⁻¹ is used for one validation TMA in order to acquire data more rapidly as a first step towards clinical translation. An undersampling ratio of two and a NB-medium apodization function was employed to transform acquired interferograms to single beam spectra. A background is collected at 120 scans per pixel at a location on the array substrate with no tissue present and used to convert sample single beams to absorbance format. Each core on the TMA is acquired separately and FT-IR images of the entire array are then compiled, analyzed, and classified using Environment for Visualizing Images (ENVI) imaging software with programs written in-house using Interactive Data Language (IDL) to perform classification and subsequent statistical analyses.

Data Analysis: Briefly, the experimental procedure involves acquiring FT-IR images and examining the resulting spectra to select features (metrics) for classification including spectral peak heights, ratios of peaks, peak areas and centers of gravity. These features capture the essential elements of the spectra, without regard to histologic tissue type or disease state. Since the number of metrics is considerably less than the number of spectral data points, this step helps reduce the dimensionality of data and decreases the time required for calculations.

The next step is to determine the probability distribution function (pdf) for each metric and quantitatively estimate the overlap of metric pdfs for different tissue classes.

Pdfs are estimated from ground truth pixels that have been marked manually by referring to a corresponding section that was H&E stained and examined by a pathologist. In general, boundary pixels are avoided to reduce systematic classification errors due to manual identification of boundaries or spectral artifacts. Large numbers of labeled pixels used for calibration likely compensates for systematic errors, biologic variation and noise. The types of classes marked by a pathologist are restricted to the task at hand. For example, the two class case in which epithelium is first segmented from stroma is described herein. Epithelial pixels are further separated into cancerous and normal classes. Each cell type (class) is denoted by a color to provide visualization.

The overlap in pdfs forms the region of ambiguity in classification and its estimate provides a preliminary estimate of the error that would result in using that specific metric for classification. The metrics are arranged in order of increasing error and employed to classify tissue. An entire classifier is built using the first metric, the first two, the first three, and so on. The total number of classifiers is equal to number of metrics that are present. The method was restricted to linear combinations or singular measures of metrics to allow interpretation of results in terms of the underlying spectral data. Statistical analysis of classification accuracy is then performed by calculating the area under the receiver operating characteristic (ROC) curve (AUC). The classification accuracy is quantitatively measured against a gold standard of tissue regions selected by a trained pathologist. Since each classifier differs from the previous by the addition of a metric, this process has also been termed the sequential forward selection process. A plot of the AUC curve for the addition of specific metrics reveals those that increase or reduce classification accuracy. Classification is then optimized by sorting the metrics by the change in the area under the ROC curve after the addition of a given metric and iterating the classification procedure. All core-level ROC values are computed by the trapezoid rule, and it is noted that this method provides a conservative estimate of the AUC since the trapezoid rule systematically underestimates the AUC obtained from a smooth curve.

The confidence of the AUC measurement is evaluated by computing a standard error, as described previously (Hanley and McNeil (1982) Radiology 143:29-36) by

$$SE ( AUC ) = \sqrt{\frac{AUC ( 1 - AUC ) + ( {n}_{1} - 1 ) ( {Q}_{1} - {AUC}^{2} ) + ( {n}_{0} - 1 ) ( {Q}_{2} - {AUC}^{2} )}{{n}_{0} {n}_{1}}} \qquad ( 1 )$$

where n₀ is the number of normal samples and n₁ is the number of cancer samples with

$${Q}_{1} = \frac{AUC}{2 - AUC}$$

$${Q}_{2} = \frac{2 {AUC}^{2}}{1 + AUC}$$

This standard error is then multiplied by a standard z-score of 1.96 to obtain a half-width for a 95% confidence interval. This method is used to assess the confidence of all AUC estimates computed for core-level ROC curves. Notably, standard error values will reduce substantially with small increases in AUC, particularly as the AUC value approaches one. Hence, higher AUC values have smaller confidence intervals for a similar sample size in sample-level studies. This technique can also be used to assess error for the AUC for pixel-level classification, but is not routinely provided employed herein since the high AUC values and large pixel numbers result in small standard errors. For example, to distinguish epithelium in tissue results in an overall AUC value of 0.9968±0.0006. Increasing the numbers of pixels beyond 50,000 had little effect on changing the AUC. Hence, while the calibration as performed on >190,000 pixels, all validation for epithelial/stromal pixels was conducted on at least 50,000 pixels in each array.

In a similar fashion, a standard error for a sensitivity or specificity value (p) for individual operating points on the AUC is calculated from the binomial approximation as

$$SE ( p ) = \sqrt{\frac{p ( 1 - p )}{n}} \qquad ( 2 )$$

where n is the number of cancer samples when p represents sensitivity and n is the number of normal samples when p represents specificity (Error! Bookmark not defined.). This formula is appropriate for any study where n×p>10, which is readily satisfied herein. These standard errors for sensitivity and specificity values are used to compute bivariate 95% confidence intervals for each point on the ROC curve to produce an overall confidence region for the ROC curve. The half-widths of these confidence intervals follow a similar pattern to the confidence interval for the AUC, with higher sensitivity and specificity values producing smaller intervals for a given sample size and larger sample sizes producing smaller intervals for a given sensitivity or specificity. Therefore, ROC curve confidence bands are very narrow for highly accurate pixel-level classification with over 50,000 spectra, and are again not visible in pixel-level ROC plots.

A general formula to approximate the 95% confidence interval for risk ratios is provided (Simel et al. (1991) J Clin Epidemiol 44:763-770) as

$$LR = {e}^{( ln \frac{{p}_{1}}{{p}_{2}} \pm {Z}_{α / 2} \sqrt{\frac{1 - {p}_{1}}{{p}_{1} {n}_{1}} + \frac{1 - {p}_{2}}{{p}_{2} {n}_{z}}} )}$$

Where for LR+, p₁=sensitivity, p₂=1—specificity, p₁ n₁ is the number of patients testing positive that are truly positive and p₂ n₂ are numbers of patients without disease testing positive. For LR-, p₁=1—sensitivity, p₂=specificity, p₁ n₁ is the number of diseased patients testing negative and p₂ n₂ are numbers of patients without disease testing negative. The statistic Z_{α/2} is calculated for α=5% for 95% confidence intervals.

### Example 2

### Molecular Contrast in Tissue Imaging

This example describes methods used to image breast cancer tissues with FT-IR.

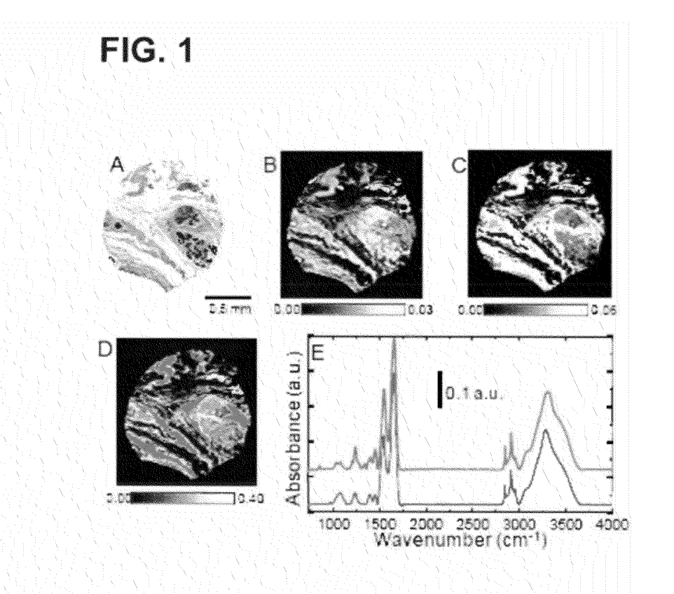

Examining tissue stained with hematoxylin and eosin (H&E) is typical in diagnostic pathology (FIG. 1A). Nucleic acid- and protein-rich regions are stained blue and pink, respectively, but the stains allow only a visualization of structure—in themselves, the stains do not directly mark tumors and follow-up recognition is required. The images in the center and on the right are generated using the molecular contrast inherent in IR imaging data from a corresponding, unstained section.

FIG. 1B quantifies the absorption commonly associated with glycoprotein- and nucleic acid-related vibrational modes (1080 cm⁻¹). The higher expression of both species is associated with secretory epithelium while FIG. 1C similarly highlights vibrational modes largely associated with stromal connective tissue (Jackson et al. (1995) Biochim Biophys Acta 1270:1-6). Pixels corresponding to stromal and epithelial cell types are highlighted by direct comparison of absorbance and H&E-stained images (FIG. 1D). These pixels can be employed to understand the underlying biochemistry, average properties, variance and differences between different cell types, disease and patient populations. Characteristic cell type spectra, obtained by averaging 190,182 pixels in 65 samples from 34 different patients, indicate that other significant biochemical differences exist between these two histologic sub-classes of tissue (FIG. 1E).

While the visualizations from IR imaging in FIG. 1 are consistent with tissue structure, they do not directly solve a clinically relevant problem. To convert molecular information to diagnostic information, automated computer algorithms were used for statistical pattern recognition (Bhargava et al. (2006) Biochim Biophys Acta 1758(7):830-845). While data handling protocols can be useful, large numbers of samples are typically needed for calibration and validation of any diagnostic technique based on biomarkers (Ransohoff (2008) J Natl Cancer Inst 100: 1419-1420). A convenient method to image large numbers of samples is to use tissue microarrays (TMAs) (Kononen et al. (1998) Nat Med 4:844-847). TMAs incorporate representative samples from many different patients on a single slide, providing both the numbers and diversity required for statistically significant classification results for spectroscopic imaging (Fernandez et al. (2005) Nat Biotechnol 23:469-474). Described below is a method that combines the use of TMAs (Camp et al. (2008) J Clin Oncol 26:5630-5637, FT-IR spectroscopic imaging (Lewis et al. (1995) Anal Chem 67:3377-3381), and automated histologic segmentation (Bhargava et al. (2006) Biochim Biophys Acta 1758(7):830-845) and apply it to breast tissue.

### Example 3

### Models for Spectral Recognition and Analysis of Class Data

Histopathologic recognition is implemented in a two-step process. As most breast cancers are epithelial in origin (May and Stroup (1991) Plast Reconstr Surg 87:193-194) and epithelial patterns are the basis for current diagnostic pathology, the tissue was first segmented into two classes—epithelium and stroma. Pixels classified as epithelium were further segmented into a cancerous or benign class.

The choice of models in entirely dependent on the desired information. Though more complex histologic models were examined (Fernandez et al. (2005) Nat

Biotechnol 23:469-474) the complexity of data analysis, number of features required and time for data classification all increase. At the same time, accuracy of complex models may be no higher than simpler models and increased effort may not be required. Hence, it was determined whether a cascaded two class model for histology [epithelium, stroma] and pathology [cancer, benign] was sufficiently effective for tumor detection. Though the importance of the stromal microenvironment in tumor development is well-recognized (Tlsty and Coussens (2006) Annu Rev Pathol: Mech Dis 1:119-150), this complexity was ignored in favor of developing a simple model. Hence, the disclosed two class model is as a screen for epithelium only, rather than to imply that all non-epithelial cell types are similar in composition or equally chemically distinct from epithelium.

Histologic Classification: The classification protocol is illustrated in FIGS. 2A-F and detailed in the methods below. Briefly, data are first acquired from a calibration TMA containing a diverse histologic, demographic and molecular profile (FIG. 2A). Next, spectral features were compared with previous reports (Fabian et al., (1995) Biospectroscopy 1:37-45; Jackson et al., (1995) Biochim Biophys Acta 1270:1-6; Petibois and Deleris (2006) TRENDS Biotechnol 24:455-462; Jackson et al. (1999) Cancer Detect Prey 23:245-253; Fabian et al. (2006) Biochim Biophys Acta 1758:874-882) and known tissue biochemistry to assess biological relevance. A total of 100 such features were selected (FIG. 2B). Features typically correspond to differential expression of chemical classes of materials; for example, glycoproteins (1080 cm⁻¹) (Jackson et al. (1999) Cancer Detect Prey 23:245-253), collagen (1236 cm⁻¹ and 1338 cm⁻¹) (Jackson et al. (1995) Biochim Biophys Acta 1270: 1-6), methylation (CH₃ asymmetric bending at 1456 cm⁻¹) and protein conformations (amide II CN stretching and NH bending at 1556 cm⁻¹) (Jackson et al. (1995) Biochim Biophys Acta 1270: 1-6). Peak heights, ratios, areas, and centers of gravity associated with these biochemical features were then constructed and termed metrics, for further evaluation.

At this stage, the goal was to reduce dimensionality of the data by fully accommodating state-of-the-art knowledge of both pathology and spectroscopy domains and not to determine if any particular metric is useful for classification. The method, further, is restricted to simple spectral measures to eventually rationalize classification results with the underlying biochemistry, providing robust assurance against artifacts or chance. An alternate classification option is to employ an “expression signature” type approach in which large spectral regions are used for segmentation.

Next, a classification protocol was developed in an integrated manner by selecting metrics useful for classification, estimating probability density functions (pdf) (FIG. 2D) and using the pdfs to predict the class of each pixel using a modified Bayesian approach (Bhargava et al. (2006) Biochim Biophys Acta 1758(7):830-845). A pixel-for-pixel comparison with a marked gold standard is used to measure accuracy qualitatively via the image (FIG. 2C) and quantitatively via the use of receiver operating characteristic (ROC) curves. Simultaneous feature selection and accuracy maximization is objectively and iteratively conducted by selecting spectral metrics to increase the area under the ROC curve (AUC).

The final protocol consists of six metrics (Table 1), which can be rapidly applied in a clinical setting. The metrics useful for epithelial segmentation largely involve relative concentrations of protein and nucleic acid, which is also the primary contrast in H&E-stained tissues. Color-coded images (FIG. 2D) are produced from the classification protocol and can be seen to correlate with conventional staining. The classified images are then used to compute spatial metrics (FIG. 2E) and the classification process is repeated to further segment epithelial pixels into cancer or normal classes. Finally, accuracy for all tasks is assessed on completely independent TMAs by the developed algorithm in which no user input is permitted.

TABLE 1
Spectral metrics selected by optimization of the histology classification
model.

| | Positions | | Molecular |
|---|---|---|---|
| Metric | (cm⁻¹) | Assignment | Origin |
| Peak Ratio | 1080:1456 | 1080 cm⁻¹: symmetric PO₂⁻ | DNA/RNA |
| | | stretching, CO stretching | Protein |
| | | 1456 cm⁻¹: asymmetric CH₃ | |
| | | bending | |
| Peak Ratio | 1556:1652 | 1556 cm⁻¹: NH bending, CN | Protein |
| | | stretching | (Amide I & |
| | | 1652 cm⁻¹: CO stretching | Amide II) |
| Peak Ratio | 1080:1238 | 1080 cm⁻¹: symmetric PO₂⁻ | DNA/RNA |
| | | stretching, CO stretching | |
| | | 1238 cm⁻¹: asymmetric PO₂⁻ | |
| | | stretching | |
| Center of | 1216-1274 | 1236 cm⁻¹: NH bending, CN | Protein |
| Gravity | | stretching, CH₂ wagging, | (Amide III) |
| | | asymmetric PO₂⁻ stretching | DNA/RNA |
| Peak Ratio | 1338:1080 | 1080 cm⁻¹: symmetric PO₂⁻ | DNA/RNA |
| | | stretching, CO stretching | Protein |
| | | 1338 cm⁻¹: CH₂ wagging | (Amide III) |
| Peak Area | 1426-1482 | 1456 cm⁻¹: asymmetric CH₃ | Protein |
| | | bending | |

### Example 4

### Validation and Robust Performance of Classifier

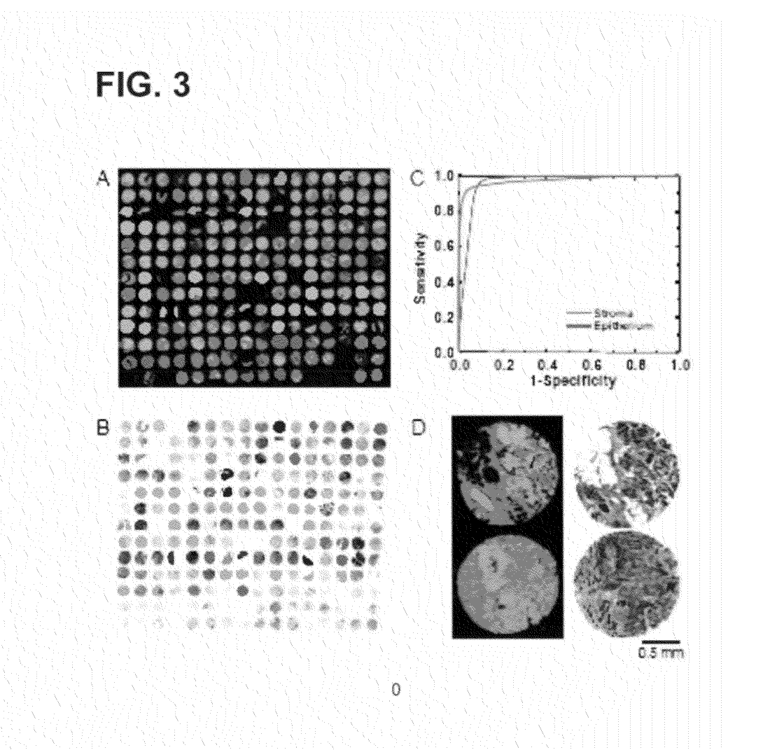

The optimal classifier yields classified images and a mean AUC of 0.996 for histologic segmentation of calibration data (FIG. 3). These results were subsequently validated on independent samples.

Validation was performed, first, on a separate section of the TMA used for calibration and, second, on additional TMAs with tissue samples from over 650 independent patients. A qualitative comparison of IR-classified (FIG. 3A) and H&E-stained (FIG. 3B) validation TMA demonstrates robust automated epithelium segmentation. Quantitative evaluation by ROC analysis (FIG. 3C and Table 2) indicates uniformly near-perfect classification in all validation sets and for different pathologies. The classification model developed on the calibration TMA readily translates to validation TMA datasets as seen in Table 2.

TABLE 2
Overview of sample sets, numbers of patients and accuracy estimates for
the two classification tasks. The first validation set (Validation TMA 1)
consists of the same patients as the calibration set.

| | | Epithelium/Stroma | Cancer/Benign |
|---|---|---|---|
| Sample Set | Samples | AUC | (AUC ± 95% CI) |
| Calibration | 65 | 0.996 | 0.95 ± .06 |
| TMA | | | |
| Validation | 77 | 0.991 | 0.97 ± 0.04 |
| TMA 1 | | | |
| Validation | 173 | 0.967 | 0.91 ± 0.04 |
| TMA 2 | | | |
| Validation | 180 | 0.948 | 0.95 ± 0.03 |
| TMA 3 | | | |
| Validation | 82 | 0.998 | 0.90 ± 0.07 |
| TMA 4 | | | |
| Validation | 87 | 0.980 | 0.93 ± 0.06 |
| TMA 5 | | | |
| Validation | 146 | 0.999 | 0.97 ± 0.03 |
| TMA 6 | | | |

The uniformly high AUC indicates that the classifier does not over-fit the spectral data and can provide reproducible results in a clinical setting. Classified images provide quick visualization of tissue structure without the necessity of adding stains or chemical dyes that irreversibly alter tissue properties (Pounder et al. (2009) Proc. SPIE 7186:71860F) while the tissue section can subsequently be used for be H&E or IHC staining as IR light is benign.

### Example 5

### Application to Cancer Segmentation

There are several potential avenues for pathologic segmentation: the first is to use the spectral data at each pixel to distinguish between cancer-bearing and benign samples. The second is to use spatial analysis of the classified image. A third approach is a combination of the two (e.g., see Bhargava et al. (2006) Biochim Biophys Acta 1758(7): 830-845).

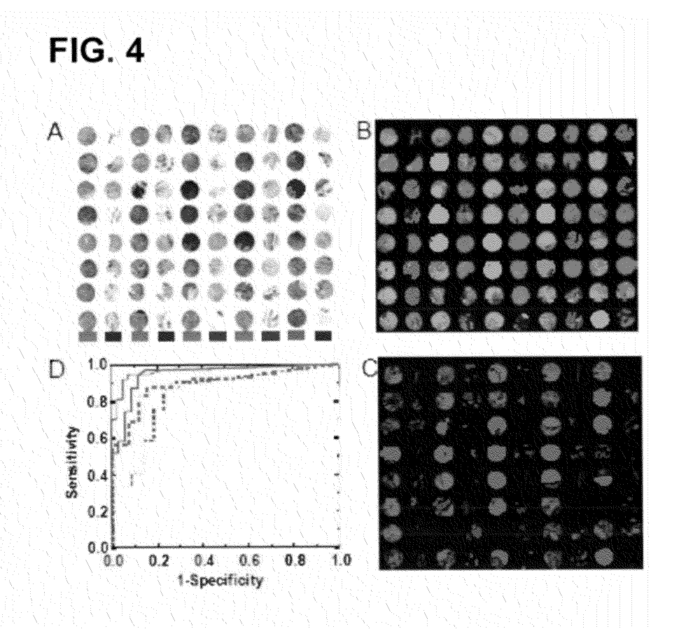

Spectral, pixel-wise cancer determination may involve measuring very small changes in chemistry, necessitating distortion-free (Davis et al. (2010) Anal Chem 82:3487-3499 and Davis et al. (2010) Anal Chem 82:3474-3486) and high signal to noise ratio data. The search for subtle spectral signals in recorded data or extensive use of numerical processing is not conducive to rapid determinations. The task here is to simply determine a tumor and its location. Breast carcinomas are identified at the most basic level as a mass of epithelial cells lacking the ductal structure, which forms the current diagnostic standard. Hence, it was determined whether forgoing the complexity of sensitive spectral changes in favor of spatial analysis would still be accurate and result in speed suitable for rapid clinical triages. Hence, spatial patterns of epithelial cells were examined as metrics. In its simplest form, the spatial segmentation captures epithelial density visible by H&E staining (FIG. 4A) and readily discerned in color-coded classified spectral images (FIG. 4B). More subtle metrics can be discerned by extracting neighborhood patterns of epithelial proliferation.

To quantify epithelial patterns around a single pixel, its spatial neighborhood is examined in progressively increasing distance for prevalence and spatial distribution of epithelial and stromal cells as well as empty space and stored as a spatial metric. At this stage, just as for spectral metrics, there is no indication of whether specific spatial metrics can provide classification. Hence, a classification protocol was developed following the procedure described previously for histologic segmentation. Classification accuracy is again assessed by ROC analysis at both the pixel and sample levels using the two class pathology model [cancer, benign]. There is a limitation here in that cores that do not contain a minimum number of pixels (<1000) are not considered in the evaluation to eliminate small, ill-processed samples and those with too little epithelium to make a diagnosis. This approach allows segmentation and can flag samples as “indeterminate” for which the protocol's diagnoses are likely to have low confidence. Here, these samples are less than 10% for any given array and are not likely to be present in larger tissue samples, e.g., from needle biopsies or surgical resections.

The developed classification protocol is highly sensitive and correctly identifies tumors in nearly all cancer-bearing samples (FIG. 4C). The confidence in individual pixels is naturally lower but each sample is assigned an overall disease state by simple majority polling. Calibration is performed on a TMA with 65 samples containing cancer and/or adjacent normal tissue from 34 patients to obtain an AUC of 0.95±0.06 (95% CI). Validation is then performed a separate TMA with 77 samples from the same patients to obtain an AUC of 0.97±0.04 (95% CI) (ROC curves in FIG. 4D). The ROC curves and 95% confidence regions, approximated using a binomial large-sample formula (Harper and Reeves (1999) Br Med J 318:1322-1323), demonstrate that this method is both sensitive and specific. The quantitative accuracy is confirmed further by ROC analysis on the independent validation set of over 650 additional patients from five additional TMAs (Table 2). The AUC values range from 0.90±0.07 (95% CI) to 0.97±0.03 (95% CI), indicating no statistically significant difference between arrays (Hanley and McNeil (1982) Radiology 143:29-36). Hence, all samples were pooled to produce an overall classification and ROC curve from 580 cancer samples and 168 normal samples (FIG. 5) with an AUC of 0.94±0.02 (95% CI).

To summarize, the validation demonstrates that the classification protocol provides accurate and reproducible results with a high level of confidence. The validation herein is exceptionally rigorous due to the order of magnitude larger samples analyzed compared to previous studies and the results are adequately powered.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A method of analyzing a breast tissue sample, comprising:
segmenting an infrared (IR) image of the breast tissue sample into epithelium and stroma, thereby classifying epithelium pixels and stroma pixels; and
segmenting epithelium pixels into cancerous or benign, thereby analyzing the sample.

2. The method of claim 1, further comprising obtaining the infrared image of the breast tissue sample. 10

3. The method of claim 2, wherein the image of the breast tissue sample is obtained using IR spectroscopic imaging instrumentation.

4. The method of claim 2, wherein the image of the breast tissue sample is obtained using Fourier transform infrared spectrometers.

5. The method of claim 1, wherein the breast tissue sample is unstained.

6. The method of claim 1, wherein the breast tissue sample is a fixed, fresh or frozen tissue sample.

7. The method of claim 1, wherein segmenting the IR image of the breast sample into epithelium and stroma comprises determining from the image one or more metrics comprising spectral peak heights, ratios of peaks, peak areas and centers of gravity.

8. The method of claim 7, wherein the spectral peak heights, ratios of peaks, peak areas and centers of gravity determined are:
a peak ratio of positions 1080:1456 cm⁻¹, 1556:1652 cm⁻¹, 1080 : 1238 cm⁻¹, and 1338:1080 cm⁻¹,
a center of gravity of position 1216-1274 cm⁻¹, and
a peak area of position 1426-1482 cm⁻¹.

9. The method of claim 7, further comprising comparing each metric to a probability distribution function (pdf) for reference epithelium and stroma.

10. The method of claim 1, wherein segmenting epithelium pixels into cancerous or benign comprises determining a spatial analysis of epithelium pixels.

11. The method of claim 10, further comprising comparing the spatial analysis of epithelium pixels and their neighborhood to a probability distribution function (pdf) for reference cancerous and benign samples.

12. The method of any of claim 1, further comprising treating a subject identified as having breast cancer.

13. The method of claim 1, further comprising selecting a subject suspected of having breast cancer and obtaining the breast tissue sample from the subject.

14. The method of claim 1, wherein the subject is a human or mammalian veterinary subject.

15. The method of claim 1, wherein the method has:
at least 95%, at least 97%, at least 98%, or at least 99% sensitivity;
at least 80% or at least 82% specificity; or
combinations thereof.

16. A computer-readable storage medium having instructions thereon for performing a method of diagnosing breast cancer, comprising:
segmenting the breast sample image into epithelium and stroma, thereby producing epithelium pixels and stroma pixels;
segmenting epithelium pixels into cancerous or benign; and
analyzing the pixels for breast cancer or benign tumor.

17. The computer-readable storage medium of claim 16, further including determining from the image one or more metrics comprising spectral peak heights, ratios of peaks, peak areas and centers of gravity in order to segment the breast sample image into epithelium and stroma.

18. The computer-readable storage medium of claim 17, wherein determining from the image one or more metrics comprises determining:
a peak ratio of positions 1080:1456 cm⁻¹, 1556:1652 cm⁻¹, 1080:1238 cm⁻¹, and 1338:1080 cm⁻¹,
a center of gravity of position 1216-1274 cm⁻¹, and
a peak area of position 1426-1482 cm⁻¹.

19. The computer-readable storage medium of claim 16, wherein segmenting epithelium pixels into cancerous or benign comprises determining a spatial analysis of the epithelium pixels.

20. The computer-readable storage medium of claim 16, further comprising comparing each metric to a probability distribution function (pdf) for reference epithelium and stroma and comparing the spatial analysis of epithelium pixels to a pdf for reference cancerous and benign samples.
